(19) United States
(12) Patent Application Publication
(10) Pub. No.: US 2024/0382713 A1
(43) Pub. Date: Nov. 21, 2024

(54) ERECTOR SPINAE REGIONAL PAIN INDWELLING CATHETER DEVICE

(71) Applicant: VANDERBILT UNIVERSITY, Nashville, TN (US)

(72) Inventors: Byron F. Stephens, Nashville, TN (US); Jeremy T. Walker, Nashville, TN (US)

(21) Appl. No.: 18/659,086

(22) Filed: May 9, 2024

Related U.S. Application Data
(60) Provisional application No. 63/502,785, filed on May 17, 2023.

(51) Int. Cl. A61M 25/00; A61M 5/142; A61M 25/02; A61M 39/10

(52) U.S. Cl. CPC A61M 25/0017; A61M 5/142; A61M 25/02; A61M 39/10; A61M 2025/0286

(57) ABSTRACT

Various embodiments of an erector spinae regional pain indwelling catheter device and method of use are described. A device can include a flexible tubular body having a wall extending a length from a proximal end to a distal end forming a main lumen with an inner diameter, a portion of the wall of flexible tubular body being fenestrated at the distal end. The device can further include a cord secured to the flexible tubular body and extending from the distal end of the flexible tubular body. The device can also include an anchor suitable for fixation to bone, where the anchor comprises an anchor body and an aperture formed in the anchor body and wherein the cord is received in the aperture formed in the anchor body to detachably connect the distal end of the flexible tubular body to the anchor.

### CROSS-REFERENCE TO RELATED APPLICATIONS

This application claims priority to, and the benefit of, U.S. Provisional Patent Application 63/502,785 entitled “Erector Spinae Regional Pain Indwelling-Catheter Device,” which was filed on May 17, 2023, and is incorporated by reference as if set forth herein in its entirety.

### BACKGROUND

The incidence of spine surgery is rapidly rising, with over 1.62 million surgeries done in the US per year. Lumbar procedures, even with less invasive techniques, often require long stays in the hospital, which increase healthcare costs. One of the primary determinants of length of stay is postoperative pain control. There is increasing pressure to perform one and even two-level lumbar fusions as an outpatient and the ability to perform these surgeries safely in an ambulatory surgery center.

### SUMMARY

Aspects of the present disclosure are related to an erector spinae regional pain indwelling catheter device and method of use. In one aspect, among others, a device can include a flexible tubular body having a wall extending a length from a proximal end to a distal end forming a main lumen with an inner diameter, a portion of the wall of flexible tubular body being fenestrated at the distal end. The device can further include a cord secured to the flexible tubular body and extending from the distal end of the flexible tubular body. The device can also include an anchor suitable for fixation to bone, where the anchor comprises an anchor body and an aperture formed in the anchor body and wherein the cord is received in the aperture formed in the anchor body to detachably connect the distal end of the flexible tubular body to the anchor. In some aspects, the cord is coupled to the wall of flexible tubular body. In some aspects, the cord is embedded in the wall of the flexible tubular body. The cord can have a cord length greater than the length of the flexible tubular body. The cord can comprise a suture material. In some aspects, the anchor comprises a biocompatable material. The anchor can also comprise a suture material. The anchor can be threaded. In some aspects, the anchor is self-driving. The flexible tubular body can be a 16 to 20 gauge catheter. The medical device can further include a luer connector secured to the proximal end of the flexible tubular body. The proximal end of the flexible tubular body can be configured to removably connect to a reservoir suitable to contain a volume of medication in liquid form.

In one or more aspects, a method, comprises forming a hole in a transverse process of a vertebrae of a subject having spinal surgery and deploying a medical device loaded on an introducer via an introducer sheath, where the medical device includes a flexible tubular body having a wall extending a length from a proximal end to a distal end forming a main lumen with an inner diameter, a portion of the wall of flexible tubular body being fenestrated at the distal end. In some aspects, the medical device further includes a cord secured to the flexible tubular body and extending from the distal end of the flexible tubular body and an anchor suitable for fixation to bone, where the anchor includes an anchor body and an aperture formed in the anchor body, and wherein the cord is received in the aperture formed in the anchor body to detachably connect the distal end of the flexible tubular body to the anchor. The method can further include embedding the anchor of the medical device into the hole formed in the transverse process, such that the fenestrated distal end of a flexible tubular body of the medical device is positioned adjacent to the anchor, and the proximal ends of the flexible tubular body and the cord extend away from the subject. Additionally, the method can include connecting the proximal end of the flexible tubular body to a reservoir containing a medication to administer the medication. In some aspects, the hole is formed by a stab incision via a drill inserted in the introducer sheath. The hole can be about 1 mm to about 3 mm in depth. The anchor can be self-driving and the hole can be formed by the insertion of the anchor. In some aspects, the proximal end of the flexible tubular body is connected to the reservoir via an infusion pump. The introducer can include a rod having a seat on a distal end and a handle on a proximal end, the seat suitable to hold the anchor of the medical device, the handle suitable to detachably secure the cord of the medical device. When the medical device is loaded on the introducer, the flexible tubular body can extend along the rod of the introducer on an opposite side from the cord such that the flexible tubular body and the cord can be slidably delivered within the introducer sheath.

### BRIEF DESCRIPTION OF DRAWINGS

Many aspects of the present disclosure can be better understood with reference to the following drawings. The components in the drawings are not necessarily drawn to scale, with emphasis instead being placed upon clearly illustrating the principles of the disclosure. In the drawings, like reference numerals designate corresponding parts throughout several views.

FIG. 1 illustrates an example of an erector spinae regional pain indwelling catheter device according to various embodiments described herein.

FIG. 2 illustrates an example placement of the erector spinae regional pain indwelling catheter of FIG. 1 and connection for administration of medication according to various embodiments described herein.

FIG. 3 illustrates an example placement of the erector spinae regional pain indwelling catheter of FIG. 1 at the transverse process of a vertebrae according to various embodiments described herein.

FIG. 4 illustrates an example delivery device of the erector spinae regional pain indwelling catheter of FIG. 1 and connection for administration of medication according to various embodiments described herein.

### DETAILED DESCRIPTION

The present disclosure relates an erector spinae regional pain indwelling catheter device to deliver local anesthetic agents during spinal and/or after surgery (e.g., fusion, laminectomy, discectomy, etc.). Spinal surgery can often result in significant postoperative pain. Pain medications are often prescribed for control of postoperative pain but can have side effects, and long-term use of such medications (e.g., opioids, etc.) lead to dependency and other issues. For some surgeries, a signal shot injection (e.g., peripheral nerve block, etc.) can be used at the time of surgery to provide a nerve block and reduce the need for additional pain medications during recovery. However, the signal shot nerve block is effective for only about a day post-operation. The erector spinae regional pain indwelling catheter device disclosed herein can provide an opioid sparing option for pain relief for a longer period during recovery.

Indwelling pain catheters are sometimes used to manage postsurgical pain after orthopedic and other surgical procedures. For example, an indwelling pain catheter can be placed under the skin of the patient, near the surgical site, to deliver anesthetic medicine at a predetermined rate to numb a nerve associated with the surgical site. Unlike other areas of the body, an effective means of achieving local blockade using indwelling pain catheters in the lumbar spine has not been previously developed. One reason for this is the difficulty in getting the catheter to stay in the correct position to block the nerves to the erector spinae, which are a source of postoperative muscle pain and spasm.

In this context, the erector spinae regional pain indwelling catheter device disclosed herein can be utilized in spine surgery to improve postoperative pain control. Some advantages of the erector spinae regional pain indwelling catheter device, among others, include lower utilization of narcotic pain medicine and better mobilization postoperatively for the patient, improving speed of recovery and reducing complications, such as deep vein thrombosis (DVT) and/or pulmonary embolism (PE). This could result in fewer days spent in the hospital, simultaneously decreasing costs for patients and increasing profit margins for the institution. Additionally, improved postoperative pain control allows for a greater ability to perform lumbar fusions as an outpatient.

According to various embodiments, the erector spinae regional pain indwelling catheter device can comprise a fenestrated catheter detachably connected to a bio-resorbable suture anchor. The erector spinae regional pain indwelling catheter device can anchor on the transverse process of a subject, a bony element of the spine extending from each side of a vertebrae. For example, the device can be positioned two or three vertebrae levels above the site of the surgery via a suture anchor. This anchor can be connected to a fenestrated catheter which then infuses local anesthetic agents (e.g., marcaine, bupivacaine, lidocaine, ±liposomal bupivacaine, and/or other agents) via an external pump. This device can remain in place for several days to a week, improving postoperative pain control. Once the pump is no longer needed, the tubing would separate from the bioabsorbable suture anchor and be removed. The anchor can also be removed.

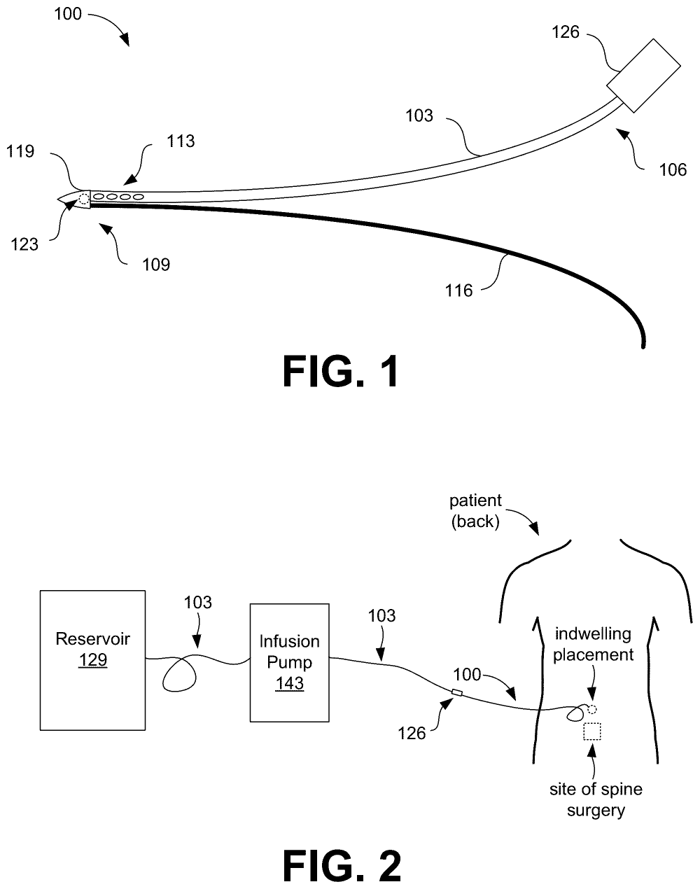

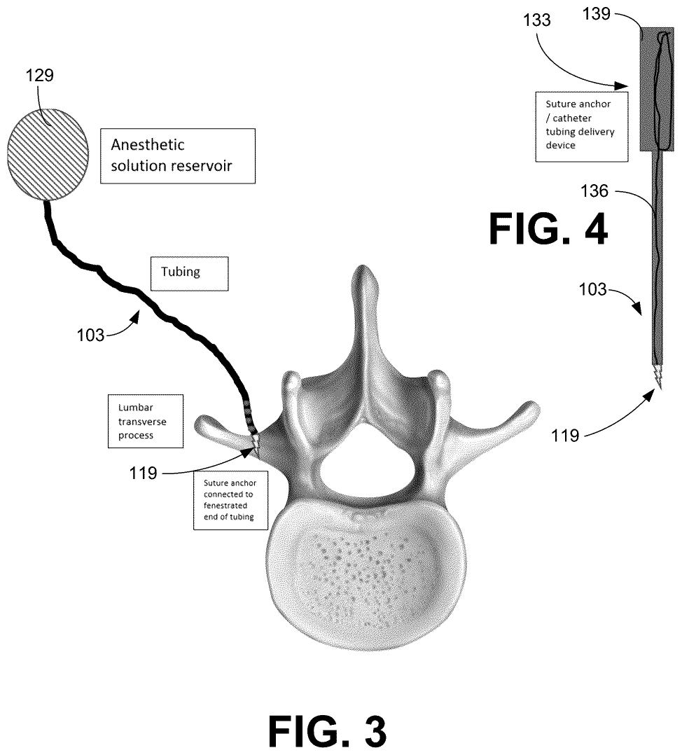

In FIGS. 1-4, the erector spinae regional pain indwelling catheter device 100 is illustrated in several examples. The erector spinae regional pain indwelling catheter device 100 is a medical device suitable for implant in a human subject and intended for short-term use to provide a perioperative and/or postoperative nerve block for pain relief related to spinal surgery. The erector spinae regional pain indwelling catheter device can be anchored to a portion (i.e. the transverse process) of a vertebra to deliver relief from pain in the erector spinae. The erector spinae regional pain indwelling catheter device 100 can be removed, including the anchor, after the course of treatment. In the event that the anchor is difficult to remove, it can be detached from the other portions of the device and remain embedded in the vertebrae of the subject.

As shown in FIG. 1, the erector spinae regional pain indwelling catheter device 100 includes tubular body 103 having a wall extending a length from a proximal end 106 to a distal end 109 and forming a main lumen with an inner diameter. According to some examples, a portion of the wall of flexible tubular body 103 is fenestrated 113 at the distal end 109. For example, the tubular body 103 can be a catheter that is fenestrated 113 at one end to deliver the medication to the designated region. The device 100 can also include a cord 116 secured to the flexible tubular body 103 and extending from the distal end 109 of the flexible tubular body 103. The device 100 can also include an anchor 119 suitable for fixation to bone. The anchor 119 may include an anchor body and an aperture 123 formed in the anchor body, such that the cord 116 is received in the aperture 123 formed in the anchor body to detachably connect the distal end 109 of the flexible tubular body 103 to the anchor 119.

The flexible tubular body 103 of the erector spinae regional pain indwelling catheter device 100 can be made of a biocompatible material, such as a plastic or polymer. For example, the tubular body 103 can be formed from a flexible and biocompatible material such as latex, silicone, Teflon, vinyl, plastic, rubber, polymer, or other material. The diameter of the flexible tubular body 103 can be provided in standard gauge sizes. For example, the flexible tubular body 103 can be a 16 to 20 gauge catheter. In some examples, the main lumen of the flexible tubular body 103 has an inner diameter in a range of approximately 0.25 mm to 4 mm. One end (e.g. distal end 109) of the flexible tubular body 103 can have a portion with fenestrations 113 or openings suitable to deliver medication and intended to be placed under the skin and near the spine of the subject having surgery. The cord 116 extends from the fenestrated end 113 to position and secure the flexible tubular body 103 adjacent to the anchor 116 for proper placement within the subject.

The opposite end (e.g. proximal end 106) of the flexible tubular body 103 can include a connector 126 suitable for leak-proof delivery of a liquid medication from a reservoir 129. The proximal end 106 of the flexible tubular body 103 is configured to removably connect to a reservoir 129 suitable to contain a volume of medication in liquid form. The connector 126 can include a fitting or lock to hold the connection together. For example, the connector 126 can be a luer connector secured to the proximal end 106 of the flexible tubular body 103. When the erector spinae regional pain indwelling catheter device 100 is implanted in the subject, the flexible tubular body 103, together with the cord 116, extend externally from the body of the subject. When connected to a reservoir 129, a portion of the flexible tubular body 103 may be looped and taped, or otherwise secured, to the skin of the subject.

The cord 116 of the erector spinae regional pain indwelling catheter device 100 can be a suture or made of a suture material. For example, the cord 116 can be made of a suture material such as nylon, polypropylene, polydioxanone, polyglycolic acid, poliglecaprone, vicryl, ethibond, fiberwire, or other absorbable or nonabsorbable suture material. The cord 116 can extend from the fenestrated end 113 of the flexible tubular body 103 such that the flexible tubular body 103 is secured to the anchor 119 for deployment. Once the anchor 119 is embedded in the vertebrae, the cord 116, together with the flexible tubular body 103, extends externally from the body of the subject. The cord 116 and flexible tubular body 103 can be held in position at the skin surface by tape or other means. The cord 116 can be coupled to the wall of flexible tubular body 103 or, in some examples, embedded in the wall of the flexible tubular body 103. The cord 116 can have a cord length equal to or greater than the length of the flexible tubular body 103.

The anchor 119 of the erector spinae regional pain indwelling catheter device 100 can be suitable for fixation to bone. The anchor 119 can be a suture anchor. The anchor 119 can include a biocompatible, nonflexible material such as a metal (e.g., titanium, stainless steel, aluminum, etc.), a ceramic, or other material. In some examples, the anchor 119 can be a composite material such as polyglycolic acid (PGA), poly-lactic acid (PLA), poly-L-lactic acid (PLLA), polyetheretherketone (PEEK), or other polymer material. In some examples, the anchor 119 can include bio-resorbable material such as, for example, a natural, synthetic, or biosynthetic polymer. In some examples, the anchor 119 can be threaded or self-driving. In some examples, the anchor 119 can be made of a suture material provided as a strip and the aperture 123 formed in the anchor body can be an elongated passage within the strip such that the cord 116 is received in the elongated passage. In some examples, the aperture 123 of the anchor 119 can be an eyelet, slit, slot, or other opening suitable to receive the cord 116. The anchor 119 can be intended to be removeable, thus removed when the device 100 is removed, however, in some implementations the anchor 119 can remain embedded in the vertebra with the cord 116 and/or anchor 119 designed to break or detach to remove the remainder of the device 100.

In some aspects, a kit can be provided including one or more erector spinae regional pain indwelling catheter devices 100 and a delivery device 133. The delivery device 133 can include an introducer suitable to receive the erector spinae regional pain indwelling catheter device 100 in a sterile environment and hold the device in position for deployment. The introducer can include a rod 136 having a seat on a distal end 109 and a handle 139 on a proximal end 106. The seat can be suitable to hold the anchor 119 of the device 100. The handle 139 can be suitable to detachably secure the cord 116 of the device. When the medical device is loaded on the introducer, the flexible tubular body 103 extends along the rod 136 of the introducer on an opposite side from the cord 116 such that the flexible tubular body 103 and the cord 116 can be slidably delivered within an introducer sheath.

The erector spinae regional pain indwelling catheter device 100 can be positioned in a subject to deliver the medication above the surgical site. For example, if the subject is having surgery at L5-S1 portion of the spine, the anchor 119 of the erector spinae regional pain indwelling catheter device 100 can be positioned in the transverse process of the L3 vertebrae of the subject. The method includes forming a hole in a transverse process of a vertebrae of a subject having spinal surgery. The hole can be formed by a stab incision via a drill inserted in the introducer sheath. For example, the hole can be about 1 mm to about 3 mm in depth.

The method also includes deploying a medical device 100 loaded on an introducer via an introducer sheath. The method also includes embedding the anchor 119 of the medical device 100 into the hole formed in the transverse process, such that the fenestrated distal end 109 of a flexible tubular body 103 of the medical device 100 is positioned adjacent to the anchor 119, and the proximal ends 106 of the flexible tubular body 103 and the cord 116 extend away from the subject. In some examples, the anchor 119 can be a self-driving and the hole can be formed by the insertion of the anchor 119.

The method also includes connecting the proximal end 106 of the flexible tubular body 103 to a reservoir 129 containing a medication to administer the medication. The proximal end 106 of the flexible tubular body 103 can be connected to the reservoir 129 directly or via an infusion pump 143.

As will be apparent to those of skill in the art upon reading this disclosure, each of the individual embodiments described and illustrated herein has discrete components and features that may be readily separated from or combined with the features of any of the other several embodiments without departing from the scope or spirit of the present disclosure. Any recited method can be carried out in the order of events recited or in any other order that is logically possible.

It is to be understood that, unless otherwise indicated, the present disclosure is not limited to particular materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

The above-described examples of the present disclosure are merely possible examples of implementations set forth for a clear understanding of the principles of the disclosure. Many variations and modifications can be made without departing substantially from the spirit and principles of the disclosure. All such modifications and variations are intended to be included herein within the scope of this disclosure and protected by the following claims.

Therefore, the following is claimed:

1. A medical device, comprising:
a flexible tubular body having a wall extending a length from a proximal end to a distal end forming a main lumen with an inner diameter, a portion of the wall of flexible tubular body being fenestrated at the distal end;
a cord secured to the flexible tubular body and extending from the distal end of the flexible tubular body; and
an anchor suitable for fixation to bone, the anchor comprising an anchor body and an aperture formed in the anchor body,
wherein the cord is received in the aperture formed in the anchor body to detachably connect the distal end of the flexible tubular body to the anchor.

2. The medical device of claim 1, wherein the cord is coupled to the wall of flexible tubular body.

3. The medical device of claim 1, wherein the cord is embedded in the wall of the flexible tubular body.

4. The medical device of claim 1, wherein the cord has a cord length greater than the length of the flexible tubular body.

5. The medical device of claim 1, wherein the cord comprises a suture material.

6. The medical device of claim 1, wherein the anchor comprises a biocompatable material.

7. The medical device of claim 1, wherein the anchor comprises a suture material.

8. The medical device of claim 1, wherein the anchor is threaded.

9. The medical device of claim 1, wherein the anchor is self-driving.

10. The medical device of claim 1, wherein the flexible tubular body is a 16 to 20 gauge catheter.

11. The medical device of claim 1, further comprising a luer connector secured to the proximal end of the flexible tubular body.

12. The medical device of claim 1, wherein the proximal end of the flexible tubular body is configured to removably connect to a reservoir suitable to contain a volume of medication in liquid form.

13. A method, comprising:
forming a hole in a transverse process of a vertebrae of a subject having spinal surgery;
deploying a medical device loaded on an introducer via an introducer sheath, the medical device comprising:
a flexible tubular body having a wall extending a length from a proximal end to a distal end forming a main lumen with an inner diameter, a portion of the wall of flexible tubular body being fenestrated at the distal end;
a cord secured to the flexible tubular body and extending from the distal end of the flexible tubular body; and
an anchor suitable for fixation to bone, the anchor comprising an anchor body and an aperture formed in the anchor body, wherein the cord is received in the aperture formed in the anchor body to detachably connect the distal end of the flexible tubular body to the anchor;
embedding the anchor of the medical device into the hole formed in the transverse process, such that the fenestrated distal end of a flexible tubular body of the medical device is positioned adjacent to the anchor, and the proximal ends of the flexible tubular body and the cord extend away from the subject; and
connecting the proximal end of the flexible tubular body to a reservoir containing a medication to administer the medication.

14. The method of claim 13, wherein the hole is formed by a stab incision via a drill inserted in the introducer sheath.

15. The method of claim 13, wherein the hole is about 1 mm to about 3 mm in depth.

16. The method of claim 13, wherein the anchor is self-driving and the hole formed by the insertion of the anchor.

17. The method of claim 13, wherein the proximal end of the flexible tubular body is connected to the reservoir via an infusion pump.

18. The method of claim 13, wherein the introducer comprises a rod having a seat on a distal end and a handle on a proximal end, the seat suitable to hold the anchor of the medical device, the handle suitable to detachably secure the cord of the medical device.

19. The method of claim 18, wherein when the medical device loaded on the introducer, the flexible tubular body extends along the rod of the introducer on an opposite side from the cord such that the flexible tubular body and the cord can be slidably delivered within the introducer sheath.

20. A kit, comprising:
at least one indwelling catheter device comprising:
a flexible tubular body having a wall extending a length from a proximal end to a distal end forming a main lumen with an inner diameter, a portion of the wall of flexible tubular body being fenestrated at the distal end;
a cord secured to the flexible tubular body and extending from the distal end of the flexible tubular body; and
an anchor suitable for fixation to bone, the anchor comprising an anchor body and an aperture formed in the anchor body, wherein the cord is received in the aperture formed in the anchor body to detachably connect the distal end of the flexible tubular body to the anchor; and
an introducer comprising a rod having a seat on a distal end and a handle on a proximal end, the seat suitable to hold the anchor of the medical device, the handle suitable to detachably secure the cord of the medical device.
